(19) United States
(12) Patent Application Publication
(10) Pub. No.: US 2018/0182493 A1
(43) Pub. Date: Jun. 28, 2018

(54) COMPARISON OF USER EXPERIENCE WITH EXPERIENCE OF LARGER GROUP

(71) Applicant: Hugh MacNaught, North Vancouver (CA)

(72) Inventor: Hugh MacNaught, North Vancouver (CA)

(21) Appl. No.: 15/903,918

(22) Filed: Feb. 23, 2018

Related U.S. Application Data
(63) Continuation-in-part of application No. 14/426,361, filed on Mar. 5, 2015.
(60) Provisional application No. 61/698,069, filed on Sep. 7, 2012.

(51) Int. Cl. G16H 50/30; G16H 50/70; G06F 17/30; G16H 10/20

(52) U.S. Cl. CPC G16H 50/30; G16H 10/20; G06F 17/30699; G16H 50/70

(57) ABSTRACT

Systems and methods for determining if a user's experience is in common with the experience of a larger number of individuals. A user's data relating to the user's lifestyle and wellness is received at a database. Candidate data, again relating to a candidate's lifestyle and wellness, is then selected from the database based on a comparison profile. The selected candidate data is analyzed in real time in conjunction with the user data. The results are then sent to the user on his or her data computing device or to someone working with the user on his of her health, wellness and lifestyle.

### RELATED APPLICATIONS

This application is a Continuation-in-Part of U.S. application Ser. No. 14/426,361 filed Mar. 5, 2015, which is a US National Stage (371) of PCT/CA2013/050687 filed Sep. 6, 2013, which claims the benefit of U.S. Provisional Application No. 61/698,069 filed on Sep. 7, 2012.

### TECHNICAL FIELD

The present invention relates to the assessment of a user's lifestyle and wellness-related data. More specifically, the present invention relates to methods, systems, and devices for assessing the user's lifestyle and wellness-related data against lifestyle and wellness-related data from others.

### BACKGROUND OF THE INVENTION

The current international epidemic of obesity and diabetes can, according to some sources, be blamed on the inactivity of people. Many of the benefits of improved wellness or health would only be possible through sustained behavior modification, and many people lack or lose motivation to persist with new regimens or activities. Those who are attempting such behavior modification, more often than not, feel that their results are not typical and that others who have attempted such measures have been more successful. This mindset can contribute to the diminution if not the elimination of the motivation to persist.

Individuals who are attempting to modify their sedentary or less active lifestyles may get disheartened if they see that their efforts are not succeeding or are not as successful as they would like. However, if such individuals can access data on individuals similar to themselves undergoing the same process, they may see that their results are typical or may even be better than expected. This sharing of data may thus increase or maintain an individual's motivation to continue with their program.

Another field in which an individual's motivation may be bolstered by knowing that others are in the same situation is women's fertility. It is known that women desiring to conceive children experience declining fertility with advancing age. Further, it is recognized that lifestyle decisions are factors influencing infertility and the inability to conceive.

Individuals who are attempting to modify their lifestyles to assist in their efforts to conceive children may get disheartened if they see that their efforts are not succeeding or are not as successful as they would like. However, if such individuals can access data on individuals similar to themselves undergoing the same process, they may see that their results are typical or may even be better than expected. This sharing of data may thus increase or maintain an individual's motivation to continue with their program. Alternatively, consulting data from others may indicate that they should consult with a medical professional regarding other approaches.

Currently, there are no systems which allow for easy sharing and comparison of lifestyle and wellness-related data between individuals who are unknown to each other, at least one of whom is in the midst of such behavior modification.

Currently, there are no systems which allow for a user to define a peer group based on personally selected or pre-selected parameters.

### SUMMARY OF INVENTION

The present invention provides systems and methods for determining if a user's experience is in common with the experience of a larger number of individuals. A user's data relating to the user's lifestyle and wellness is received at a database. Candidate data, again relating to a candidate's lifestyle and wellness, is then selected from the database based on a comparison profile. The comparison profile can be automatically recommended by the system, or be manually created by the user using specific shared user profiles or keywords that match other profiles. The selected candidate data is analyzed in real time in conjunction with the user data. The results are then sent to the user on his or her data computing device or to someone working with the user on his or her health, wellness, and lifestyle.

In a first aspect, the present invention provides a method for determining whether a user's lifestyle and wellness are common among a plurality of other individuals, the method comprising:
a) receiving user data relating to lifestyle and wellness, said user data being gathered from said user;
b) determining a comparison profile;
c) accessing a database of database data, said database of database data containing data for said plurality of other individuals;
d) selecting candidate data from said database, said candidate data being related to lifestyle and wellness and said candidate data being data for individuals matching at least a predetermined portion of said comparison profile;
e) analyzing said candidate data selected in step d) and said user data;
f) sending results of said analysis to a destination device.

In a second aspect, the present invention provides a system for analyzing data relating to lifestyle and wellness, the system comprising:
a database for storing user data and candidate data, said user data and candidate data both relating to lifestyle and wellness;
a data processor for processing said user data and said candidate data;

wherein said system is used in a method for determining whether a user's experiences relating to lifestyle and wellness are common among a plurality of other individuals, the method comprising:
a) receiving said user data, said user data being gathered from said user;
b) determining a comparison profile;
c) accessing said database, said database containing said candidate for said plurality of other individuals;
d) selecting said candidate data from said database, said specific candidate data being data relating to lifestyle and wellness for individuals matching at least a predetermined portion of said comparison profile;
e) analyzing said specific candidate data selected in step d) and said user data;
f) sending results of said analysis to said user.

In a third aspect, the present invention provides a system for comparing a user's experiences relating to lifestyle and wellness with experiences for a plurality of other individuals, the system comprising:
a database server containing a database of database data, said database of database data containing data relating to lifestyle and wellness for said plurality of other individuals;
a server configured for:
   receiving user data relating to lifestyle and wellness, said user data being gathered from said user;
   determining a comparison profile;
   accessing said database of database data;
   selecting candidate data from said database, said candidate data being related to lifestyle and wellness for individuals matching at least a predetermined portion of said comparison profile;
   analyzing said candidate data and said user data;
   sending results of said analysis to a destination;
      wherein said server is in data communications with said database server.

In yet a further aspect, the present invention provides computer readable medium having encoded thereon computer readable and computer executable instructions which, when executed, implements a method for determining whether a user's experiences relating to lifestyle and wellness are common among a plurality of other individuals, the method comprising:
a) receiving user data, which relates to lifestyle and wellness, said user data being gathered from said user;
b) determining a comparison profile;
c) accessing a database of database data, said database of database data containing data relating to lifestyle and wellness for said plurality of other individuals;
d) selecting candidate data from said database, said candidate data being data relating to lifestyle and wellness for individuals matching at least a predetermined portion of said comparison profile;
e) analyzing said candidate data selected in step d and said user data;
f) sending results of said analysis to a destination device.

### BRIEF DESCRIPTION OF THE DRAWINGS

The embodiments of the present invention will now be described by reference to the following figures, in which identical reference numerals in different figures indicate identical elements and in which:

FIG. 1 is a block diagram of a system according to one aspect of the invention; and

FIG. 2 is a flowchart detailing the steps in a method according to another aspect of the invention.

### DETAILED DESCRIPTION OF THE INVENTION

As noted above, the provision of information comparing a user's results data to results data from their peers would provide additional motivation to users since they could get a better idea of whether their experience was typical or not.

Such feedback could be presented to show how the individual ranked within a self-defined or pre-defined population of interest. The data may be collected through manual entry (using a questionnaire or a survey), automated entry from a single device, automated entry from multiple devices, or manual or automated entry of data from multiple devices. Such devices may include sensors designed to measure heart rate, weight, activity, blood glucose, or galvanic skin response as well as sensors which gather other lifestyle and wellness-related data from the user. Data from these and other devices are collected and analyzed using unique algorithms designed to perform a multivariate or meta-analysis and may be used to track and compare one or more parameters over time against a self- or pre-defined population. These are then used to generate an overall wellness or status measure.

It should be noted that, for the purposes of this document, the term lifestyle and wellness-related data is defined as including medical or health-related data. While the discussion in this document will focus on the sharing of lifestyle and wellness-related data, the concept of sharing personal experience/data and using a database of shared experience/data for comparison with one's own experience/data may be used in other fields. The present invention can be applied to any information for which a user wishes to assess his or her own personal experience against the experience of a larger number of people. One example would be the assessment of fuel consumption for a particular model and year of car to enable the owner to understand whether their experience was typical of real-life experience of similar owners, and how this compares to manufacturers' claims. Other examples relating to health, physical and mental wellness may include comparing a user's lifestyle and wellness data against athletes or against the general populace. Similarly, the invention may be used in comparing athletic results against data collected from a selected group of candidates (the candidate data). As an example, an athlete may wish to compare his physical wellness data or his lifestyle data against candidates who are top-level athletes such as those who have competed in the Olympic Games. A further example may be that of a user comparing his lifestyle (perhaps including diet information, exercise information, and physical characteristics) against those who are in the same age/weight/physical activity level as himself. Of course, such a user may also wish to compare his lifestyle data with data for top-level athletes as well. In a further example, a user who is undergoing a specific dietary regime (e.g. a high protein/low carbohydrate diet, a reduced sugar diet, etc.) may wish to compare the results of his diet against the results of others also undergoing the same regimen.

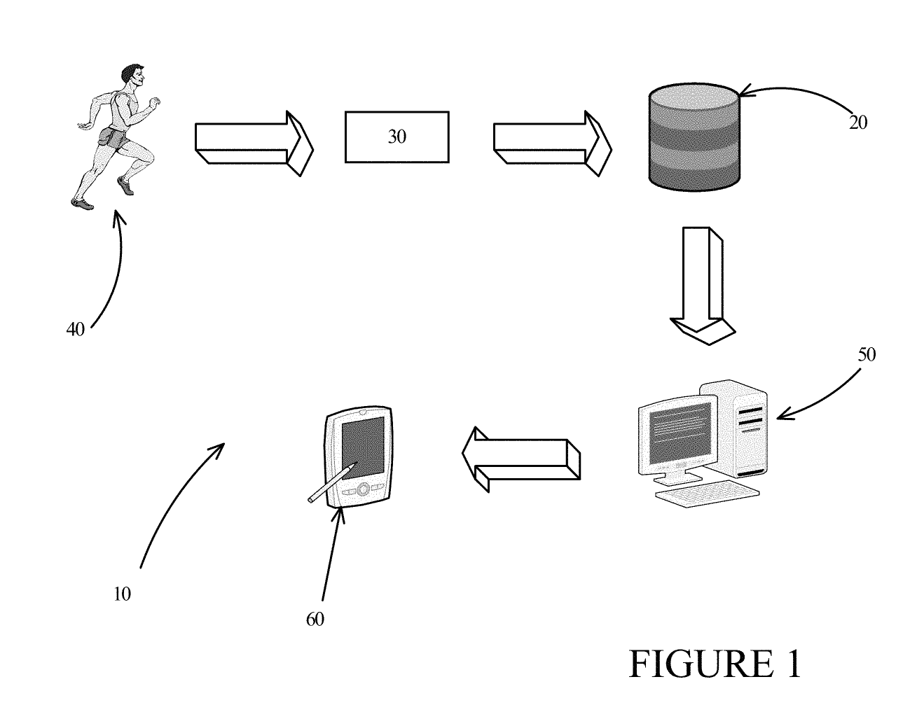

Referring to FIG. 1, a block diagram of a system which uses the invention is illustrated. The system 10 has a database 20 which receives data from data source 30. The data in the data source 30 are derived from the user 40. The data in the database 20 and the data from the data source 30 are then analyzed by data processor 50. The results of the analysis are then transmitted to a data processing device 60. The data processing device 60 may be operated or used by user 40 or by a person who is working with the user on the user's health and wellness (e.g. a doctor or personal trainer).

The database 20 may take the form of a computer server with suitable data storage capabilities and communications capabilities such that it can communicate with other devices. The data source 30 may be sensors attached to devices that measure the user's lifestyle and wellness-related data. As an example, sensors attached to a heart rate monitor, a blood pressure measuring, or other devices for measuring the user's vital signs may function as the data source 30. Alternatively, the data source 30 may take the form of questionnaires or surveys filled out by the user. The data in the questionnaires or surveys are then entered into a computing device and then sent to the database 20. Another potential data source 30 may be another database such a hospital or doctor's database. A further alternative data source 30 may be a database at a health club.

It should be noted that, once the user lifestyle and wellness-related data is received, it is added to the database so that it can be used when other users are searching the database. To protect a user's privacy, the user lifestyle and wellness-related data saved in the database can be anonymized while retaining the relevant datapoints in the database. As an example, any identifying data can be removed while still retaining the user's physical characteristics. As an alternative to the anonymizing the user data in the database, the user data may be encrypted prior to storage with only the user being in possession of the encryption key which would be required to decrypt the user data. The user can then control who or what can access his or her data by providing or withholding the encryption key as he or she sees fit.

The data processor 50 may take the form of a server coupled to the database 20. Depending on the configuration of the system, the server used as the data processor 50 may be the same server that contains the database 20. Alternatively, the data processor 50 and the database 20 may be geographically separate. In cloud-based implementations of the system, the database may be on servers geographically remote from servers operating as the data processor.

The data processing device 60 may be any data processing device such as a tablet, a personal computer, a smartphone, or any other device which may be used to present data or information to a user. It should be clear that the bulk if not all of the processing of the user data and the candidate data is performed by the data processor 50 in FIG. 1. The data processing device 60 would mainly serve as a destination device that receives the results of the processing of both the user data and the candidate data. As noted above, the data processing device may be operated by the user or by someone working with the user such as a doctor, personal trainer, or a suitable health, wellness, or fitness worker.

Regarding the processing of the user lifestyle and wellness-related data (data derived from the user) and the candidate lifestyle and wellness-related data (the data in the database that are from other users or other individuals whose data is available), the processing may involve a number of factors and may take a number of different forms.

Processing may involve multivariate comparisons, statistical analyses, meta-analyses, and other analytical processes. As well, processing may involve tracking both candidate data and user data over time. In addition, this tracking may involve tracking correlations, differences, and any other comparisons between the user data and the candidate data. The processing may, depending on the configuration, produce a wellness or status measure for the user based on the comparison of the user data with the candidate data. The processing can be performed in real-time to give the user the processing results with as little delay as possible. Such processing would ensure up-to-date data on the database. Users can then use the latest up-to-date candidate data in processing or comparing their own user data.

Alternatively, processing may not be overly complicated. The processing may be as simple as a direct comparison between the user lifestyle and wellness-related data and the candidate lifestyle and wellness-related data, with similar data points or readings being compared to determine if the user lifestyle and wellness-related data is within expected bounds relative to the candidate lifestyle and wellness-related data. As an example, the user's blood pressure reading may be directly compared with the blood pressure reading for individuals in the database whose parameters (age, physical parameters, activity level, physical condition) correspond to those of the user. Thus, a male user who is 37 years old, weighing 180 lbs., exercises moderately three times a week but who is diabetic can have his blood pressure readings compared with candidate lifestyle and wellness-related data in the database from male individuals who are between the ages of 35-39, weighing 170-190 lbs., who exercise between 2-4 times a week, and who are diabetic. The 37 year old user can then determine if his blood pressure readings are within the range of the blood pressure readings for this group whose parameters are similar to his. Of course, the same user may also wish to compare his lifestyle and wellness-related data against the lifestyle and wellness-related data for individuals who are slight younger or slightly older than him.

The processing may also involve photographic or video processing. As an example, a user may take a digital photograph of himself or of an aspect of himself (e.g. a lesion on his skin or a wound or the user's skin after the application of a specific lotion or cream). The digital photograph can then be uploaded to the database and compared with other digital photographs of similar subjects. Alternatively, the digital photograph may be used to derive data regarding the user (e.g. extrapolating or deriving the user's height or weight from a full body photograph) or regarding the subject of the digital photograph (e.g. size or color of a lesion or wound or the color/condition of the user's skin after the lotion or cream is applied). The digital photographs can be periodically updated and the characteristics derived from the digital photographs or the photographs themselves can be tracked through time to provide a time-based record for comparison with earlier photographs or photographs from other users.

To determine which lifestyle and wellness-related data are to be retrieved from the database and are to be compared/processed with the user lifestyle and wellness-related data, a comparison profile can be used. A comparison profile would detail the parameters to be used in selecting candidate lifestyle and wellness-related data from the database. If specific candidate data indicates that the person from whom the data was derived does not meet the comparison profile, then that candidate data is not selected. On the other hand, if the candidate data indicates that the person from whom the candidate data was derived does meet the comparison profile, then that candidate data is selected. The comparison profile may, depending on the configuration, be automatically generated or it may be manually generated by a user or by someone acting with the user (e.g. the user's doctor, personal trainer, etc.). The comparison profile may include parameters such as age, height, weight, level of physical activity, blood pressure, blood sugar level, pre-existing physical conditions (e.g. diabetes, hypertension, vision impairment, etc.), hair color, eye color, gender, and any other health, physique, appearance, or physical condition parameter. For cases where women as desiring to compare their physical situation with others who might be trying to conceive, the comparison profile (as well as the user data and candidate data) may include biochemical markers (e.g. hormone concentrations), start of menstruation, duration of menstruation, physical characteristics of body fluids, and galvanic skin response. For clarity, the parameters in the comparison profile may use ranges in combination with specific values. Thus, the comparison profile may look for candidate data for individuals having a specific blood pressure range but whose eyes are specifically colored hazel.

As noted above, the comparison profile may be user-selected or, depending on the configuration, be automatically generated. Thus, a user's user data may, by default, be automatically compared with candidate data for individuals in the user's age/health condition group. Alternatively, the comparison profile may be created to select candidate data for individuals who are from a select and very specific group as defined by the comparison profile. Users can then use the comparison profile to sift through potentially large amounts of candidate data to find data that meets the user's very specific needs. Any parameter relating to health, lifestyle, or wellness may be used in the comparison profile. In addition to the parameters already mentioned above, these parameters may include: socioeconomic factors, familial traits or factors, geographic location, interests, or other factors which are not typically associated with a given classification.

After the processing has been completed, the results are then sent to a destination device. The results may be sent to the user's device of choice (e.g. personal computer, tablet computer, or smartphone) or to a data processing device at a facility used by the user (e.g. clinic, hospital, health club, or gym). It should be noted that the results may be presented in any format usable by the data processing device. As such, the results may be sent in table format, as pure data, or in XML or HTML format.

As noted above, the comparison of physical traits and/or physiological conditions/traits between users and data in the database may be for various purposes. The comparison may be for women who are trying to conceive, individuals who are trying to lose weight, or, indeed anyone who wishes to compare their efforts at lifestyle changes with others in the same situation.

Data may be automatically gathered from the user or from candidates using many different methods. User data may thus be automatically gathered using any of the following:
a heart rate monitor,
a blood pressure monitor,
automated data inputs masked to an end user (e.g. date, time, weather etc.),
a cardiopulmonary fitness device such as stationary bike, treadmill, etc. that provides data for load, resistance, speed, incline,
an accelerometer fitness device that provides data relating to range of movement, number of movements per time interval,
a data stream captured through a Bluetooth or a WiFi connection to a smart phone or tablet,
available data from medical or health records (e.g. cholesterol, blood glucose, electrolyte levels, etc.)

Alternatively, user data may be gathered manually and entered manually into the system. For such manual data gathering, the user may be required to login to a user portal on the system and manually enter data such as demographic information. Depending on the configuration of the system, a web portal or web interface may be provided for user access. Assuming the system is accessible online, the user may login by way of a suitable smartphone, a suitable tablet device, or any suitable device for online access.

In another implementation, the system may be cloud-based with data being streamed to a cloud-based storage subsystem. The data may be analyzed using both proprietary and well-known algorithms to perform multivariate analysis. For more involved analyses, neural networks and deep learning methods may be used with the invention. Once analyses have been performed, the system may then stream results to a user's suitable smartphone or tablet or the results may be viewable by way of a web interface. Preferably, reports are customized to enable real-time reporting of results based on parameters defined by the user.

It should be clear that the reports provided to the user may have many variants. A report using a proprietary index may be used or a report giving an overall comparison/ranking may also be used. As well, a custom ranking based on user preferences may be used. Such user preferences may include age, gender, height, weight, ethnic background, geographical location, membership or inclusion in a club or organisation, dietary preferences (e.g. vegetarian) etc. A user can thus choose to compare his or her data against others whose characteristics are user selected.

In one example, the system of the present invention may be used to determine a user's condition relative to others. For this example, the user characteristic being compared is heart rate recovery (HRR). HRR following maximal exercise is a predictor of mortality and is a useful indicator of progress for people including those afflicted with cardiometabolic disorders. Many of these individuals will improve their HRR results, or results measured in part using HRR, through regular exercise training but may suffer loss of motivation because of a perception of little result or progress.

Such a user may be equipped with a wearable heart rate monitor that uploads real time data to the system. In addition to the wearable monitor, an interfaced treadmill capable of reporting speed and inclination may be used. Finally, user inputs, including user demographic information as well as parameters defining the user's preferred peer group, would be used.

With the above components in place, the methodology begins with the user using the treadmill. The user starts the treadmill using a constant speed of 5.1 km/h and an incline of 0 percent. After one minute, the incline is increased by 2 percent. Every minute thereafter, the incline is increased by 1 percent. The user must maintain the pace and continue to increase the incline until they are exhausted and cannot continue. At that point, the user needs to discontinue the test and the time elapsed is recorded. At 5 minutes post exercise, the user's heart rate is recorded. The decline from the maximum heart rate during the test is then calculated.

The calculated decline is directly correlated with cardiopulmonary fitness, and users may wish to better understand their performance or standing relative to those whom they consider peers based on the most current real-time data.

The system generated report then provides the user's performance using a proprietary CPQ (cardiopulmonary quotient) index. The system concurrently calculates maximal oxygen uptake using an algorithm with data from the heart monitor and the exercise device. The system also calculates estimated metabolic equivalents (METs) using another algorithm and system data. The algorithms include data such as date, time of day and weather.

Results can be presented to the user based on standardized gender and age categories, or alternative classifications defined by the user (e.g. age, location, gender, height, weight, medically relevant conditions and habits such as whether the candidate is a smoker or is diabetic, etc.).

As an example, a user may self describe as female, East Indian, age 35, smoker, have a calculated body mass index (BMI) that is categorised as obese, and be diagnosed as being in a pre-diabetic state. The user will be able to periodically assess her CPQ using the system and monitor her results and compare them in real time against a peer group defined by the preceding parameters rather than using static tables or charts. This provides greater assurance that the reported results are based on the best available current information and that they are reflective of current societal trends and norms.

The present invention performs analyses and its reports provide guidance or conclusions regarding the user's physical or mental condition. The present invention not only reports raw data regarding the user's performance relative to a user selected group but it also provides a metric (e.g. the CPQ index) that is a result of real time analysis. This metric incorporates data points that, to the user, will be obvious (e.g. heart rate) and non-obvious (e.g. daylight hours, temperature).

It should be clear that the present invention provides an alternative to the incorrect and inaccurate data and conclusions based on BMI. Body mass index (BMI) is simply calculated on the basis of sex, height and weight and is based on very old tables. There are a number of known deficiencies with the use of BMI yet its use persists because no one has built anything useable to replace it. Further, BMI and the tables it is based on make no attempt to consider a number of factors that many people would find important to their self-perception and identification. Finally, much has occurred in society since BMI was first introduced and a BMI-based assessment does not provide much insight into the evolution of dietary and exercise habits.

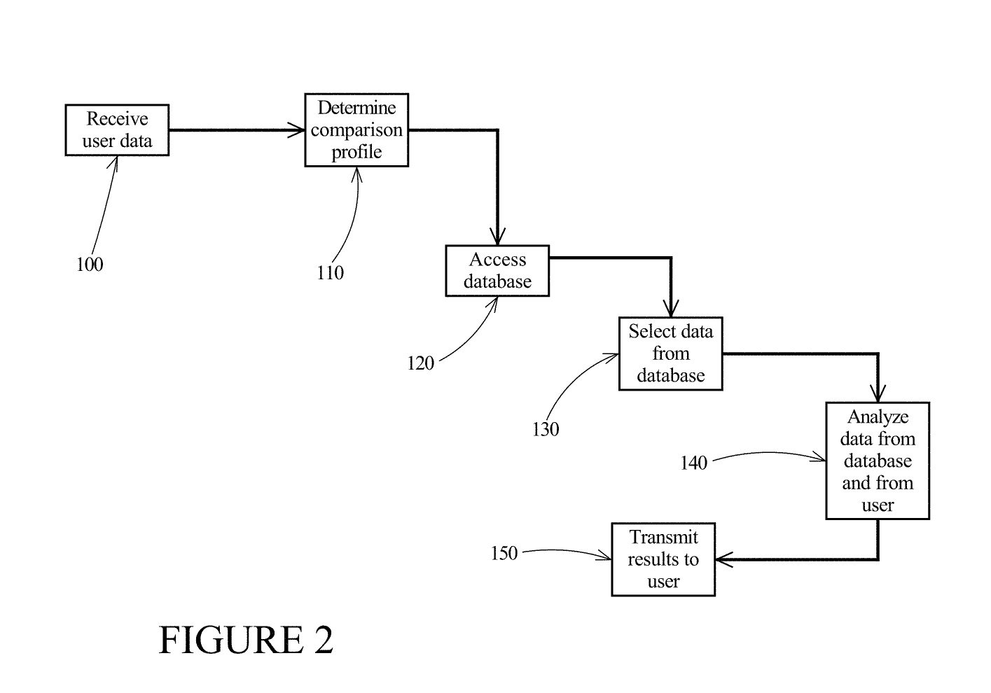

The steps in a method according to one aspect of the invention are illustrated in the flowchart of FIG. 2. Step 100 is that of receiving user lifestyle and wellness-related data from a data source. The lifestyle and wellness-related data may be received automatically or it may be transmitted from the data source manually. As noted above, the user data may come from questionnaires or surveys manually completed by the user, or it may be automatically gathered from sensors coupled to the user.

Step 110 is that of determining a comparison profile. The comparison profile may be created by the user or by someone working with the user. Alternatively, the comparison profile may be a default comparison profile. Such a default comparison profile may be set to a profile that matches the user's profile and may be derived from the user data.

Once the comparison profile has been determined, the database is then accessed in step 120. The database is searched for candidate data which matches at least part of the comparison profile (step 130). Of course, depending on the configuration, candidate data which are selected from the database may match all, some, or just part of the comparison profile. The match for the comparison profile may be based on the profile of the individual from whom the candidate profile was derived or the match may be based on the actual candidate data.

With the relevant candidate data now selected, the candidate data and the user data are then analyzed (step 140). As noted above, the analysis may involve anywhere from a simple comparison to complex statistical and multivariate analysis. After the analysis is done, the results are then sent to a data processing device used by the user or someone working with the user (step 150). It should be noted that the data processing device which receives the results may be user operated or operated by a health/fitness/wellness worker working with the user. In one variant, the user can designate which device receives the results of the processing.

The method steps of the invention may be embodied in sets of executable machine code stored in a variety of formats such as object code or source code. Such code is described generically herein as programming code, or a computer program for simplification. Clearly, the executable machine code may be integrated with the code of other programs, implemented as subroutines, by external program calls or by other techniques as known in the art.

The embodiments of the invention may be executed by a computer processor or similar device programmed in the manner of method steps, or may be executed by an electronic system which is provided with means for executing these steps. Similarly, an electronic memory means such computer diskettes, CD-ROMs, Random Access Memory (RAM), Read Only Memory (ROM) or similar computer software storage media known in the art, may be programmed to execute such method steps. As well, electronic signals representing these method steps may also be transmitted via a communication network.

Embodiments of the invention may be implemented in any conventional computer programming language. For example, preferred embodiments may be implemented in a procedural programming language (e.g.“C”) or an object oriented language (e.g.“C++”). Alternative embodiments of the invention may be implemented as pre-programmed hardware elements, other related components, or as a combination of hardware and software components. Embodiments can be implemented as a computer program product for use with a computer system. Such implementations may include a series of computer instructions fixed either on a tangible medium, such as a computer readable medium (e.g., a diskette, CD-ROM, ROM, or fixed disk) or transmittable to a computer system, via a modem or other interface device, such as a communications adapter connected to a network over a medium. The medium may be either a tangible medium (e.g., optical or electrical communications lines) or a medium implemented with wireless techniques (e.g., microwave, infrared or other transmission techniques). The series of computer instructions embodies all or part of the functionality previously described herein. Those skilled in the art should appreciate that such computer instructions can be written in a number of programming languages for use with many computer architectures or operating systems. Furthermore, such instructions may be stored in any memory device, such as semiconductor, magnetic, optical or other memory devices, and may be transmitted using any communications technology, such as optical, infrared, microwave, or other transmission technologies. It is expected that such a computer program product may be distributed as a removable medium with accompanying printed or electronic documentation (e.g., shrink wrapped software), preloaded with a computer system (e.g., on system ROM or fixed disk), or distributed from a server over the network (e.g., the Internet or World Wide Web). Of course, some embodiments of the invention may be implemented as a combination of both software (e.g., a computer program product) and hardware. Still other embodiments of the invention may be implemented as entirely hardware, or entirely software (e.g., a computer program product).

A person understanding this invention may now conceive of alternative structures and embodiments or variations of the above all of which are intended to fall within the scope of the invention as defined in the claims that follow.

We claim:

1. A system for comparing a user's experiences relating to lifestyle and wellness with experiences for a plurality of other individuals, the system comprising:
a database server containing a database of database data, said database of database data containing data relating to lifestyle and wellness for said plurality of other individuals;
a server configured for:
receiving user data relating to lifestyle and wellness, said user data being gathered from said user;
determining a comparison profile;
accessing said database of database data;
selecting candidate data from said database, said candidate data being related to lifestyle and wellness for individuals matching at least a predetermined portion of said comparison profile;
analyzing said candidate data and said user data;
sending results of said analysis to a destination;
wherein said server is in data communications with said database server.

2. The system according to claim 1, wherein said server is configured to compare said candidate data with said user data.

3. The system according to claim 1, wherein said server creates said comparison profile based on a profile of said user.

4. The system according to claim 1, wherein said server creates said comparison profile based on input from said user.

5. The system according to claim 1, wherein said user data is received from sensors coupled to said user.

6. The system according to claim 1, wherein said user data is derived from at least one questionnaire completed by said user.

7. The system according to claim 1, wherein said user data and said candidate data includes at least one of:
heart rate,
weight,
level of physical activity,
blood glucose level,
diet related data,
temperature,
biochemical markers,
start of menstruation,
duration of menstruation,
physical characteristics of body fluids,
galvanic skin response.

8. The system according to claim 1, wherein said server is configured to determine a comparison over time between said user data and said candidate data previously selected.

9. The system according to claim 1, wherein said server is configured to add said user data to said database.

10. The system according to claim 1, wherein said server is configured to remove identity data from said candidate data prior to analyzing said candidate data.

11. A computer readable medium having encoded thereon computer readable and computer executable instructions which, when executed, implements a method for determining whether a user's experiences relating to lifestyle and wellness are common among a plurality of other individuals, the method comprising:
a) receiving user data which relates to lifestyle and wellness, said user data being gathered from said user;
b) determining a comparison profile;
c) accessing a database of database data, said database of database data containing data relating to lifestyle and wellness for said plurality of other individuals;
d) selecting candidate data from said database, said candidate data being data relating to lifestyle and wellness for individuals matching at least a predetermined portion of said comparison profile;
e) analyzing said candidate data selected in step d) and said user data;
f) sending results of said analysis to a destination device.
